(19) United States
(12) Patent Application Publication
(10) Pub. No.: US 2023/0235366 A1
(43) Pub. Date: Jul. 27, 2023

(54) PROCESS FOR THE BIOLOGICAL PRODUCTION OF HYDROGEN AND/OR METHANE BY ABSORPTION AND BIOLOGICAL CONVERSION OF CARBON DIOXIDE

(71) Applicants: BIOREWEAL S.R.L., TORTORETO (IT); BIORENOVA SOCIETA' PER AZIONI, MONTORIO AL VOMANO (IT)

(72) Inventors: Michele GALIANO, BOLOGNA (IT); Riccardo REVERSO, CREMONA (IT)

(21) Appl. No.: 17/999,757

(22) PCT Filed: May 21, 2021
(86) PCT No.: PCT/EP2021/063588

§ 371 Date: Nov. 23, 2022

(30) Foreign Application Priority Data
Jun. 1, 2020 (IT) 102020000013006

(51) Int. Cl. C12P 3/00; C12P 5/02

(52) U.S. Cl. CPC C12P 3/00; C12P 5/023

(57) ABSTRACT

A process for the biological production of hydrogen and/or methane by absorption and biological conversion of carbon dioxide, includes the steps of being performed by co-culture of one or more hydrogen-producing bacteria in at least one first reactor, and one or more acetogenic bacteria in at least one second reactor, and/or one or more methanogenic microorganisms in at least one third reactor.

### TECHNICAL FIELD

The present disclosure relates to a process for the production of hydrogen and/or methane starting from carbon dioxide (CO₂).

### BACKGROUND

In recent years, the release of gaseous emissions into the atmosphere, particularly those with a high carbon dioxide content, has become a problem that is felt increasingly both by public opinion and by governments and institutions tasked with dealing with environmental issues. The concentration of carbon dioxide in the atmosphere, which is the main responsible for climate change phenomena worldwide, is increasing constantly mainly due to the increase in combustion activities and the reduction of the capacity of our planet to absorb carbon dioxide, due to deforestation and the reduction in zoo- and phytoplankton, organisms with the ability to regulate ecological chains.

For the above reasons, the reduction of greenhouse gases (GHGs) is currently one of the global priorities to contain the increase in the Earth's temperature within the limit set by the 2015 Paris Agreement on climate. Among greenhouse gases (GHGs), carbon dioxide is the priority issue; in fact, in 2018, CO₂ emissions from combustion accounted for approximately 70% of total global greenhouse gas emissions (“The Emissions Gap Report 2019”, UN Environment Programme).

Despite continued warnings from the scientific and academic world, carbon dioxide emissions continue to rise. The latest data presented in the annual report of the Global Carbon Project indicate that at the end of 2019 CO₂ emissions reached a new record of 36.8 billion metric tons globally, while the concentration in the atmosphere continues to remain at values steadily above 400 ppm since 2016 (“Global Carbon Budget 2019”, Global Carbon Project).

These levels of concentration represent a clear sign of the inability of oceans and forests to absorb the continuous increases in the emission of carbon dioxide, which, by remaining “trapped” in the atmosphere, facilitates the rise in global temperature.

In order to avoid this increase and the inevitable catastrophic consequences on our planet, it is therefore absolutely necessary to adopt a joint series of actions aimed both at reducing emissions and at an effective absorption of carbon dioxide, in order to facilitate the progressive reduction of the quantities that have led to the current record levels of concentration in the atmosphere.

With specific reference to the first aspect, it is evident that in order to achieve an effective and substantial reduction in climate-changing gas emissions, it is necessary to act on the energy sector, not only with actions aimed at containing consumption but also and above all by introducing new ways of generating energy with reduced emission of climate-changing gases.

In this regard, the production of methane by biological means, by virtue of the action of methanogenic microorganisms, is a procedure known in the background art and already performed in various plants for the treatment of solid and liquid waste, as well as in so-called “power to gas” (PtG) plants. However, in the former, the production of methane through fermentation of materials having a very complex and non-homogeneous composition, as is indeed waste, is always accompanied by parallel fermentations that, by lowering the methane yield due to the production of undesirable gases such as CO₂, NOₓ, SO₂, etc., make it necessary to adopt systems for the purification of biogas into biomethane (“upgrading”), with a consequent impact on investment and operating costs.

In PtG plants, on the other hand, the hydrogen necessary for the reaction of conversion of carbon dioxide into methane is produced by electrolysis, i.e., a process with a high absorption of electric power.

EP2016/077771 also describes the production of methane by biological conversion of carbon dioxide performed by symbiosis between one or more methanogenic microorganisms and: (i) one or more hetero-autotrophic cyanobacteria and/or microalgae, or (ii) one or more sulfobacteria and/or acetobacteria. In EP2016/077771, the specified symbiotic interaction is characterized, by its very nature, by a complex management which inevitably also affects the production potential of the microorganisms involved. Moreover, in EP2016/077771 the quantities of input carbon dioxide in the process are strictly linked and limited to the reaction of methanogenesis, i.e., the biological conversion of said carbon dioxide performed by the methanogenic microorganisms exclusively for the purpose of methane production. Finally, in EP2016/077771, since the production of methane is simultaneous with the biological conversion of carbon dioxide, the molecular hydrogen flow produced in the symbiosis with the hetero-autotrophic cyanobacteria and/or microalgae, or with the sulfobacteria and/or acetobacteria, is exclusively intended for the production of methane.

In this context, it seems useful to point out that hydrogen is the known fuel with the highest heating value per unit mass and its combustion does not produce carbon dioxide and other emissions harmful to humans and to the environment.

Hydrogen is already used extensively in various industrial applications and its demand is growing continuously: from 20 million tons in 1975 it has reached over 70 million tons in 2018 (The Future of Hydrogen 2019, IEA).

However, currently hydrogen is generated almost entirely through the thermochemical conversion of fossil fuels, such as methane and coal (so-called “gray hydrogen”). This production process facilitates the generation of hydrogen at relatively low costs, but entails a large consumption of non-renewable resources and high emissions of carbon dioxide into the atmosphere.

Other hydrogen production methods provide for the use of electric power or thermal energy. In particular, electrolytic processes, such as high-temperature electrolysis, used to release the hydrogen contained in water, use electric power and thermal energy (so-called “green hydrogen”). These processes have the advantage of not producing carbon dioxide in the hydrogen generation phase, but entail high costs associated with the consumption of electric power.

As regards the treatment of carbon dioxide, too, there are numerous projects under development and already known in the background art which are aimed at capturing and storing CO₂ underground (CCS—Carbon Capture and Storage). These technologies are much debated for the actual potential and opportunities offered, as well as for the risks entailed, especially in terms of safety of the storage sites.

### SUMMARY

In view of the problems described above, the aim of the present disclosure is therefore to provide a process for the absorption and biological conversion of carbon dioxide which is complementary with respect to capture techniques and which, by overcoming the limitations of storage techniques, provides for the use of CO₂ as a raw material.

The disclosure provides a process for the production of hydrogen that is not intended exclusively for the reaction of conversion of carbon dioxide into methane and in particular a process for the production of hydrogen by biological means, with reduced energy consumption and, therefore, convenient and sustainable from an economic and environmental point of view.

The disclosure also provides a process for the production of methane by biological conversion of carbon dioxide or exhaust gases containing carbon dioxide that allows to obtain methane with a higher yield and efficiency than the processes known so far, characterized by a high degree of purity and, therefore, by a reduced content of unwanted gases.

Moreover, the present disclosure provides a process which, while removing carbon dioxide and producing hydrogen and/or methane, also makes it possible to obtain biological material, organic acids and minerals to be used in the agricultural, food, pharmaceutical and industrial sector.
the disclosure further provides a process for the biological production of hydrogen and/or methane by absorption and biological conversion of carbon dioxide which is highly reliable and flexible in application, is relatively easy to provide and has competitive costs and almost no process waste.

This aim and these and other advantages that will become better apparent hereinafter are achieved by providing a process for the biological production of hydrogen and/or methane by absorption and biological conversion of carbon dioxide, said process comprising the steps of:
(i) introducing carbon dioxide in at least one first reactor containing up to 95% by volume of a first culture medium comprising one or more hydrogen-producing bacteria and keeping under continuous stirring in anaerobic conditions until a stationary phase of the growth of the one or more hydrogen-producing bacteria is achieved, obtaining a first fermented culture medium and a gaseous mixture of hydrogen and residual carbon dioxide, wherein the one or more hydrogen-producing bacteria are selected from the group consisting of Clostridium beijerinckii, Clostridium butyricum, Clostridium bifermentans, Clostridium sporogenes, Rhodobacter sphaeroides, Rhodobacter capsulatus, Enterobacter cloacae, Thermotoga neapolitana and Hungateiclostridium thermocellum;
(ii) optionally separating the hydrogen from the gaseous mixture of hydrogen and residual carbon dioxide obtained in step (i);
(iii) introducing the gaseous mixture of hydrogen and residual carbon dioxide obtained in step (i) in at least one of:
   a) at least one second reactor comprising up to 95% by volume of a second culture medium which comprises one or more acetogenic bacteria and keeping under continuous stirring in anaerobic conditions, obtaining a second fermented culture medium and hydrogen, and
   b) at least one third reactor comprising up to 95% by volume of a third culture medium comprising one or more methanogenic microorganisms and keeping under continuous stirring in anaerobic conditions, obtaining a third fermented culture medium and a gaseous mixture comprising methane,
or
introducing the residual carbon dioxide separated from the hydrogen in step (ii) in the at least one second reactor which comprises up to 95% by volume of a second culture medium comprising one or more acetogenic bacteria and keeping under continuous stirring in anaerobic conditions, obtaining a second fermented culture medium;
wherein the one or more acetogenic bacteria are selected from the group consisting of Acetoanaerobium noterae, Acetoanaerobium pronyense, Acetoanaerobium sticklandii, Acetobacterium carbinolicum, Moorella thermoacetica, Butyribacterium methylotrophicum, Eubacterium limosum, Moorella thermoautotrophica, Desulfosporosinus orientis and Blautia producta; and
the one or more methanogenic microorganisms are selected from the group consisting of Methanolacinia paynteri, Methanothermobacter wolfeii, Methanothermobacter thermautotrophicus, Methanothermobacter marburgensis, Methanosarcina barkeri, Methanosarcina mazei, Methanobacterium bryantii, Methanothermobacter tenebrarum and Methanosarcina thermophila.

### DETAILED DESCRIPTION OF THE DISCLOSURE

Further characteristics and advantages of the disclosure will become better apparent from the following detailed description.

The process according to the disclosure seeks to contribute to the reduction of the concentration of carbon dioxide in the atmosphere, while producing hydrogen and/or methane, i.e., important energy resources, by means of a co-culture of specific bacteria and microorganisms that allows to achieve high levels of production efficiency.

For the production of hydrogen, the process according to the disclosure uses one or more of the following hydrogen-producing bacteria, preferably, but not exclusively the strains identified in brackets by respective deposit numbers:

Clostridium beijerinckii (ATCC No. 25752 and ATCC No. 17778), Clostridium butyricum (ATCC No. 860 and ATCC No. 19398), Clostridium bifermentans (ATCC No. 19299, NCTC No. 1340 and NCTC No. 8780), Clostridium sporogenes (ATCC No. 3584 and ATCC No. 19494), Rhodobacter sphaeroides (ATCC No. 17023), Rhodobacter capsulatus (ATCC No. 11166), Enterobacter cloacae (IIT-BT No. 08), Thermotoga neapolitana (ATCC No. 49049) and Hungateiclostridium thermocellum (ATCC No. 27405).

For the absorption of carbon dioxide, the process according to the disclosure uses instead one or more of the following acetogenic bacteria, preferably, but not exclusively, the strains identified in brackets by respective deposit numbers:

Acetoanaerobium noterae (ATCC No. 35199), Acetoanaerobium pronyense (DSM No. 27512), Acetoanaerobium sticklandii (DSM No. 519), Acetobacterium carbinolicum (DSM No. 2925), Moorella thermoacetica (ATCC No. 39073, ATCC No. 49707 and ATCC No. 35608), Butyribacterium methylotrophicum (DSM No. 3468 and ATCC No. 33266), Eubacterium limosum (ATCC No. 8486), Moorella thermoautotrophica (ATCC No. 33924), Desulfosporosinus orientis (DSM No. 765) and Blautia producta (ATCC No. 27340).

Said acetogenic bacteria culture can also be used for the absorption of carbon dioxide present in gaseous mixtures that originate from other industrial processes.

For the production of methane, the process according to the disclosure uses one or more of the following methanogenic microorganisms, preferably, but not exclusively, the strains identified in brackets by the respective deposit numbers:

Methanolacinia paynteri (DSM No. 2545), Methanothermobacter wolfeii (ATCC No. 43096), Methanothermobacter thermautotrophicus (DSM No. 3720 and ATCC No. 29096), Methanothermobacter marburgensis (DSM No. 2133), Methanosarcina barkeri (ATCC No. 43569), Methanosarcina mazei (ATCC No. 43573), Methanobacterium bryantii (ATCC No. 33272), Methanothermobacter tenebrarum (DSM No. 23052) and Methanosarcina thermophila (DSM No. 2980).

In each of the reactors used in the process of the present disclosure, a quantity up to 95% of the total volume of each reactor of culture medium is added with the nutritional components required for the one or more bacteria and microorganisms belonging to the groups described above.

The nutritional components suitable for the above cited bacteria and microorganisms are those known to the person skilled in the art; for example the hydrogen producing bacteria can be grown in: Reinforced clostridial medium (RCM), Rhodospirillaceae medium available on the German Collection of Microorganisms and Cells (DSMZ)—catalogue number DSMZ 27, Nutrient agar available on the German Collection of Microorganisms and Cells (DSMZ)—catalogue number DSMZ 1; acetogenic bacteria can be grown in: Nutrient agar available on the German Collection of Microorganisms and Cells (DSMZ)—catalogue number DSMZ 1, Thermotoga TF(C) medium—available on the German Collection of Microorganisms and Cells (DSMZ)—catalogue number DSMZ 613, Clostridium noterae medium available on the America Type Culture Collection (ATCC)—catalogue number ATCC 1344, Moorella medium available on the German Collection of Microorganisms and Cells (DSMZ)—catalogue number DSMZ 60, Modified chopped meat medium available on the America Type Culture Collection (ATCC)—catalogue number ATCC 1490, Desulfovibrio (Postgate) medium available on the German Collection of Microorganisms and Cells (DSMZ)—catalogue number DSMZ 63; methanogenic microorganisms can be grown in: Modified chopped meat medium available on the America Type Culture Collection (ATCC)—catalogue number ATCC 1490, Methanosarcina barkeri medium available on the German Collection of Microorganisms and Cells (DSMZ)—catalogue number DSMZ 120a., Methanogenium medium available on the German Collection of Microorganisms and Cells (DSMZ)—catalogue number DSMZ 141.

In each reactor, the fermentation continues by appropriately controlling temperature, the pH, and the supply of nutrients and microelements, as known to the person skilled in the art.

Step (i) of the process starts with the introduction of carbon dioxide in the head space of the at least one first reactor.

In the process according to the present disclosure, in fact, carbon dioxide (CO₂) is used as raw material. Therefore, gaseous emissions that are rich in carbon dioxide but also include other gaseous components must undergo pretreatment before being dispatched to absorption and/or biological conversion according to the process described herein. The pretreatment, necessary to separate the carbon dioxide from any other gaseous components and to purify it from the presence of any pollutants, can be performed by using various known technologies for capturing CO₂ such as, by way of non-limiting example, membrane separation, so-called pressure swing adsorption, and washing with amines.

Preferably, in step (i) the operating temperature and pressure of the at least one first reactor are respectively lower than 40° C. and lower than 250 kPa (2.5 bar).

Optionally, the process according to the disclosure can comprise the step ii) of separating the hydrogen from the gaseous mixture of hydrogen and residual carbon dioxide obtained in step (i) by using known technologies suitable for this purpose.

Step (iii) of the process starts with the introduction of the gaseous mixture of hydrogen and residual carbon dioxide obtained in step (i) in the at least one second reactor and/or in the at least one third reactor.

Preferably, in step (iii) the operating temperature and pressure of the at least one second reactor are respectively lower than 39° C. and lower than 250 kPa (2.5 bar).

Preferably, in step (iii) the operating temperature and pressure of the at least one third reactor are respectively lower than 75° C. and lower than 500 kPa (5.0 bar).

In a preferred embodiment of the process according to the disclosure, after reaching the stationary growth phase of the one or more hydrogen-producing bacteria, step (i) comprises the additional steps of:
(i.a) drawing the gaseous mixture of hydrogen and residual carbon dioxide from the head space of the at least one first reactor;
(i.b) unloading from the at least one first reactor a volume of the first fermented culture medium until a concentration of the one or more hydrogen-producing bacteria in the first fermented culture medium of no less than 2 g/l is reached;
(i.c) loading inside the at least one first reactor a quantity by volume of the first culture medium that is equal to the volume of the first fermented culture medium unloaded in step (i.b);
(i.d) restarting the growth of the one or more hydrogen-producing bacteria until the stationary growth phase of the one or more hydrogen-producing bacteria is reached and repeating steps (i.a) to (i.c).

Within the scope of this embodiment, the process of the disclosure preferably further comprises the step (i.b′) of separating the first fermented culture medium unloaded in step (i.b) into a liquid component and a solid component.

Separation of the liquid component from the solid component is performed by unloading the fermented culture medium into an adapted separation device, such as for example a decanter centrifuge. Said fermented culture medium may be used for the extraction of organic acids to be used in the food, agricultural and/or pharmaceutical sector. The solid component is constituted by bacteria which can be used in the food, agricultural and/or pharmaceutical sector or as nutrients for subsequent fermentations. From the liquid component it is instead possible to recover water to be reused for the preparation of culture media.

Optionally, the gaseous mixture of hydrogen and residual carbon dioxide obtained in step (i) is drawn from the first reactor and stored in one or more accumulation tanks.

In one embodiment of the process according to the disclosure, in step (iii) the gaseous mixture of hydrogen and residual carbon dioxide obtained in step (i) is introduced in at least one between the at least one second reactor and the at least one third reactor.

Preferably, in step (iii) the introduction of the gaseous mixture of hydrogen and residual carbon dioxide in at least one between the at least one second reactor and the at least one third reactor occurs, preferably continuously, by injecting the gaseous mixture into the second culture medium and/or into the third culture medium.

In another embodiment, the process according to the disclosure comprises the step of (ii) separating the hydrogen from the gaseous mixture of hydrogen and residual carbon dioxide obtained in step (i), wherein in step (iii) the residual carbon dioxide separated from the hydrogen in step (ii) is introduced in the at least one second reactor. Preferably, the hydrogen separated from the residual carbon dioxide in step (ii) is introduced in one or more accumulation tanks.

Unlike what occurs for the at least one first reactor of step (i), the unloading of the fermented medium from the at least one second and/or at least one third reactor of step (iii) is not correlated with the growth cycles of the bacteria and microorganisms, but rather with the need to keep constant the volume of culture medium within the at least one reactor used in step (iii).

The fermented culture medium of the at least one second reactor of step (iii) may be unloaded into a suitable device for separating the liquid component from the solid component, such as for example a decanter centrifuge. Said fermented culture medium may be used for the extraction of organic acids and/or minerals to be used in industry and/or in the food and/or pharmaceutical sector. From the liquid component it is instead possible to recover water to be reused for the preparation of culture media.

The solid component is constituted by bacteria that can be used in the agricultural sector or as nutrients for subsequent fermentations.

In the process according to the present disclosure, the production of methane (“methanization”) occurs by the action of methanogenic microorganisms, which use CO₂ and produce methane according to the following reaction:

4H₂+CO₂--->CH₄+2H₂O; i.e.,

4H₂+HCO⁻+H⁺--->CH₄+3H₂O

Methanogenic microorganisms are able to perform this reaction by coupling the oxidation of molecular hydrogen (H₂) with the reduction of CO₂ (final electron acceptor) with reoxidation of NAD by virtue of the continuous removal of said H₂. The absorption of CO₂ by methanogenic microorganisms in the at least one third reactor is limited to use in the methanogenesis reaction according to the above cited reaction. In the process of methanogenesis according to the disclosure, the hydrogen needed by the methanogenic microorganisms is produced in the at least one first reactor and the gaseous mixture of hydrogen and carbon dioxide obtained in step (i) is conveyed into the at least one third reactor directly, or after a step of storage in accumulation tanks.

The gaseous mixture, which comprises methane produced at the end of the methanization step, is in turn drawn from the head space of the at least one third reactor, optionally in a continuous mode, and further purified or stored in one or more accumulation tanks.

The process according to the disclosure, therefore, allows to obtain hydrogen and/or methane depending on whether in step iii) the gaseous mixture obtained in step i) is introduced only in the at least one second reactor which comprises acetogenic bacteria, only in the at least one third reactor which comprises methanogenic microorganisms, or in both.

When the mixture of hydrogen and residual carbon dioxide is introduced, by injection into the culture medium directly or after storage, a gaseous mixture comprising methane is produced in the at least one third reactor assigned to methanization.

The fermented culture medium of the at least one third reactor of step (iii) may be unloaded into a suitable device for separating the liquid component from the solid component, such as for example a decanter centrifuge. The solid component is constituted by microorganisms to be used in the agricultural sector or as nutrients for subsequent fermentations. From the liquid component it is instead possible to recover water to be reused for the preparation of the culture media.

The process according to the disclosure allows furthermore to purify the methane from the gaseous mixture obtained from the at least one reactor assigned to methanization.

In a preferred embodiment, the process according to the disclosure further comprises the step of:

(iv) introducing the gaseous mixture comprising methane obtained in step (iii) in at least one additional second reactor which comprises up to 95% by volume of a culture medium which comprises one or more acetogenic bacteria and keeping under continuous stirring in anaerobic conditions, obtaining a fermented culture medium and methane, wherein the one or more acetogenic bacteria are selected from the group consisting of the acetogenic bacteria described above.

Preferably, in said step (iv) the operating temperature and pressure of the at least one additional second reactor are respectively lower than 39° C. and lower than 250 kPa (2.5 bar).

In one embodiment, the process according to the disclosure further comprises the step of introducing in the at least one second reactor and/or in the at least one third reactor carbon dioxide that originates from sources which are external with respect to the one that originates from step (i), preferably in continuous mode by injecting the carbon dioxide into the culture media.

Advantageously, the process according to the disclosure, unlike other processes such as for example the symbiotic process described in EP2016/077771, does not limit the introduction of carbon dioxide into the process to only the quantities necessary for conversion into methane, but also allows its absorption, increasing the potential of the process according to the disclosure to contribute to the reduction of the concentration of carbon dioxide in the atmosphere.

The carbon dioxide, in fact, is introduced in a virtuous process of circular economy which allows to transform a problem of global importance into resources, i.e., hydrogen and methane of biological origin, produced with the utmost respect for environmental sustainability and with reduced energy consumption.

The disclosures in Italian Patent Application No. 102020000013006 from which this application claims priority are incorporated herein by reference.

1-14. (canceled)

15. A process for the biological production of hydrogen and/or methane by absorption and biological conversion of carbon dioxide, said process including the following steps:
(i) introducing carbon dioxide in at least one first reactor containing up to 95% by volume of a first culture medium comprising one or more hydrogen-producing bacteria and keeping under continuous stirring in anaerobic conditions until a stationary phase of the growth of the one or more hydrogen-producing bacteria is achieved, obtaining a first fermented culture medium and a gaseous mixture of hydrogen and residual carbon dioxide, wherein the one or more hydrogen-producing bacteria are selected from the group consisting of Clostridium beijerinckii, Clostridium butyricum, Clostridium bifermentans, Clostridium sporogenes, Rhodobacter sphaeroides, Rhodobacter capsulatus, Enterobacter cloacae, Thermotoga neapolitana and Hungateiclostridium thermocellum;
(ii) optionally separating the hydrogen from the gaseous mixture of hydrogen and residual carbon dioxide obtained in step (i);
(iii) introducing the gaseous mixture of hydrogen and residual carbon dioxide obtained in step (i) in at least one of:
a) at least one second reactor comprising up to 95% by volume of a second culture medium which comprises one or more acetogenic bacteria and keeping under continuous stirring in anaerobic conditions, obtaining a second fermented culture medium and hydrogen, and
b) at least one third reactor comprising up to 95% by volume of a third culture medium comprising one or more methanogenic microorganisms and keeping under continuous stirring in anaerobic conditions, obtaining a third fermented culture medium and a gaseous mixture comprising methane,
or
introducing the residual carbon dioxide separated from the hydrogen in step (ii) in the at least one second reactor which comprises up to 95% by volume of a second culture medium comprising one or more acetogenic bacteria and keeping under continuous stirring in anaerobic conditions, obtaining a second fermented culture medium;
wherein the one or more acetogenic bacteria are selected from the group consisting of Acetoanaerobium noterae, Acetoanaerobium pronyense, Acetoanaerobium sticklandii, Acetobacterium carbinolicum, Moorella thermoacetica, Butyribacterium methylotrophicum, Eubacterium limosum, Moorella thermoautotrophica, Desulfosporosinus orientis, and Blautia producta; and
the one or more methanogenic microorganisms are selected from the group consisting of Methanolacinia paynteri, Methanothermobacter wolfeii, Methanothermobacter thermautotrophicus, Methanothermobacter marburgensis, Methanosarcina barkeri, Methanosarcina mazei, Methanobacterium bryantii, Methanothermobacter tenebrarum, and Methanosarcina thermophila.

16. The process according to claim 15, wherein in step (i) the operating temperature and pressure of the at least one first reactor are respectively lower than 40° C. and lower than 250 kPa.

17. The process according to claim 15, wherein in step (iii) the operating temperature and pressure of the at least one second reactor are respectively lower than 39° C. and lower than 250 kPa.

18. The process according to claim 15, wherein in step (iii) the operating temperature and pressure of the at least one third reactor are respectively lower than 75° C. and lower than 500 kPa.

19. The process according to claim 15, wherein after reaching the stationary growth phase of the one or more hydrogen-producing bacteria step (i) includes the following steps:
(i.a) drawing the gaseous mixture of hydrogen and residual carbon dioxide from the head space of the at least one first reactor;
(i.b) unloading from the at least one first reactor a volume of the first fermented culture medium until a concentration of the one or more hydrogen-producing bacteria in the first fermented culture medium of no less than 2 g/l is reached;
(i.c) loading inside the at least one first reactor a quantity by volume of the first culture medium that is equal to the volume of the first fermented culture medium unloaded in step (i.b); and
(i.d) restarting the growth of the one or more hydrogen-producing bacteria until the stationary growth phase of the one or more hydrogen-producing bacteria is reached and repeating steps (i.a) to (i.c).

20. The process according to claim 19, further comprising the step of (i.b′) separating the first fermented culture medium unloaded in step (i.b) into a liquid component and a solid component.

21. The process according to claim 15, wherein the gaseous mixture of hydrogen and residual carbon dioxide obtained in step (i) is drawn from the first reactor and stored in one or more accumulation tanks.

22. The process according to claim 15, wherein in step (iii) the gaseous mixture of hydrogen and residual carbon dioxide obtained in step (i) is introduced in at least one between the at least one second reactor and the at least one third reactor.

23. The process according to claim 15, including the step of (ii) separating the hydrogen from the gaseous mixture of hydrogen and residual carbon dioxide obtained in step (i), wherein in step (iii) the residual carbon dioxide separated from the hydrogen in step (ii) is introduced in the at least one second reactor.

24. The process according to claim 15, wherein in step (iii) the introduction of the gaseous mixture of hydrogen and residual carbon dioxide in at least one between the at least one second reactor and the at least one third reactor occurs, preferably continuously, by injecting the gaseous mixture into the second culture medium and/or into the third culture medium.

25. The process according to claim 15, further including the step of:
(iv) introducing the gaseous mixture comprising methane obtained in step (iii) in at least one additional second reactor which comprises up to 95% by volume of a culture medium which comprises one or more acetogenic bacteria and keeping under continuous stirring in anaerobic conditions, obtaining a fermented culture medium and methane, wherein the one or more acetogenic bacteria are selected from the group consisting of the acetogenic bacteria.

26. The process according to claim 25, wherein in step (iv) the operating pressure and temperature of the at least one additional second reactor are respectively lower than 39° C. and lower than 250 kPa.

27. The process according to claim 15, further including the step of separating the second fermented culture medium and/or the third fermented culture medium into a liquid component and a solid component.

28. The process according to claim 15, further including the step of introducing in the at least one second reactor and/or in the at least one third reactor carbon dioxide that originates from sources which are external with respect to the one that originates from step (i).
